(19) United States
(12) Patent Application Publication
(10) Pub. No.: US 2022/0163527 A1
(43) Pub. Date: May 26, 2022

(54) Methods of Screening and Compounds for Adverse Response to an Implant

(71) Applicant: PXD Limited, Newcastle upon Tyne (GB)

(72) Inventor: David Langton, Newcastle upon Tyne (GB)

(21) Appl. No.: 17/434,868

(22) PCT Filed: Mar. 2, 2020
(86) PCT No.: PCT/GB2020/050497

§ 371 Date: Aug. 30, 2021

(30) Foreign Application Priority Data
Mar. 1, 2019 (GB) 1902779.6

(51) Int. Cl. G01N 33/569; G01N 33/564

(52) U.S. Cl. CPC G01N 33/56977; G01N 33/564; G01N 2800/52; G01N 2800/50; G01N 2800/24

(57) ABSTRACT

Methods of screening a subject to determine whether or not they are at risk of having an adverse reaction to metal debris (ARMD), also known as adverse local tissue reaction (ALTR), methods of screening compounds for use in preventing or ameliorating such an adverse response and compounds for use in treating a subject.

The present invention relates to methods of screening a subject to determine whether or not they are at risk of having an adverse reaction to metal debris (ARMD), also known as adverse local tissue reaction (ALTR), methods of screening compounds for use in preventing or ameliorating such an adverse response and compounds for use in treating a subject. An example of such a reaction includes an aseptic lymphocyte dominated vasculitis associated lesion (ALVAL). In particular, the present invention relates to determining whether or not a subject is at risk of having an adverse reaction to an implant.

Hip replacement or hip arthroplasty is a procedure in which the hip joint is replaced by a prosthetic implant. A total hip replacement consists of replacing the acetabulum and the femoral head while hemiarthroplasty only replaces the femoral head.

Metal on metal (MoM) hip replacements were reintroduced globally at the turn of the century (Treacy R B, McBryde C W, Pynsent P B. Birmingham hip resurfacing arthroplasty. A minimum follow-up of five years. J Bone Joint Surg Br. 2005; 87(2):167-70). With promise of increased stability (lower risk of dislocation) and a reduction in wear, they quickly gained popularity with surgeons throughout the world (12th Annual Report. National Joint Registry of England and Wales. 2015.). Intended to be more durable than conventional plastic hips, they were implanted mainly into younger patients in order to allow them to return to as active a life as possible (McMinn D, Daniel J. History and modern concepts in surface replacement. Proc Inst Mech Eng H. 2006; 220(2):239-51).

Unfortunately, complications began to emerge. This was primarily due to adverse immune responses that patients developed to metal debris (primarily composed of cobalt and chromium particles) which was generated from the hips (Pandit H, Glyn-Jones S, McLardy-Smith P, Gundle R, Whitwell D, Gibbons C L, et al. Pseudotumours associated with metal-on-metal hip resurfacings. J Bone Joint Surg Br. 2008; 90(7):847-51).

There appear to be two general cellular responses (Natu S, Sidaginamale R P, Gandhi J, Langton D J, Nargol A V. Adverse reactions to metal debris: histopathological features of periprosthetic soft tissue reactions seen in association with failed metal on metal hip arthroplasties. J Clin Pathol. 2012; 65(5):409-18.). With massive metal exposure, the predominant cellular response is macrophagic, and the resulting injury is mostly limited to the bone.(10) With intermediate levels of metal exposure, a cellular response (known as aseptic lymphocyte dominated vasculitis associated lesion (“ALVAL”)(11) can develop alongside the macrophagic infiltration. ALVAL is associated with the development of massive fluid effusions and widespread soft tissue necrosis (Langton D J, Jameson S S, Joyce T J, Hallab N J, Natu S, Nargol A V. Early failure of metal-on-metal bearings in hip resurfacing and large-diameter total hip replacement: A consequence of excess wear. J Bone Joint Surg Br. 2010; 92(1):38-46). The risk factors for the development of ALVAL are poorly understood.

Unfortunately, ARMD/ALTR(Langton D J, Joyce T J, Jameson S S, Natu S, Holland J P, Nargol A V F, De Smet K. Adverse reaction to metal debris following hip resurfacing the influence of component type, orientation and volumetric wear. Journal of Bone and Joint Surgery (Br) 2011; 93:566; Langton D J, Jameson S S, Joyce T J, Natu S, Nargol A V F. Early failure of metal-on-metal bearings in hip resurfacing and large-diameter total hip replacement. A CONSEQUENCE OF EXCESS WEAR. Journal of Bone and Joint Surgery (Br) 2010; 92-B; and, Pseudotumours associated with metal-on-metal hip resurfacings. Pandit H, Glyn-Jones S, McLardy-Smith P, Gundle R, Whitwell D, Gibbons C L, Ostlere S, Athanasou N, Gill H S, Murray DW. J Bone Joint Surg Br. 2008 July; 90(7):847-51) is not limited to metal on metal devices (Jacobs J J, Cooper H J, Urban R M, Wixson R L, Della Valle C J. What do we know about taper corrosion in total hip arthroplasty? J Arthroplasty. 2014; 29(4):668-9). Over the last decade, there has been a global trend towards the adoption of the use of larger diameter bearings as standard, irrespective of the bearing combination (ie metal on plastic, ceramics) (12th Annual Report. National Joint Registry of England and Wales. 2015). During this time period, however, the support structure to accommodate these large diameter heads (the male taper/trunnion) was reduced in size (Langton D J, Sidaginamale R, Lord J K, Nargol A V, Joyce T J. Taper junction failure in large-diameter metal-on-metal bearings. Bone Joint Res. 2012; 1(4):56-63) We have observed several patients who have suffered severe reactions from this interface—the “taper junction” (Langton D J, Jameson S S, Joyce T J, Gandhi J , Sidaginamale R, Mereddy P, et al. Accelerating failure rate of the ASR total hip replacement. J Bone Joint Surg Br. 2011; 93(8):1011-6).

The Applicants believe that there will be a global issue with early failure of joint replacements over the next ten years. The Applicants have thus determined that the identification of patients most at risk of developing ARMD/ALTR would be beneficial in order to streamline patient surveillance and/or consider alternative approaches in certain cases to avoid potentially catastrophic patient reactions. It may also facilitate the development of therapeutic interventions.

In accordance with the present invention, there is provided a method of screening a subject to determine the likelihood of adverse response to metal debris (ARMD) or adverse local tissue reaction (ALTR), said method comprising the steps of:

i) contacting a subject's biological sample with means to detect the presence and/or absence of a marker associated with an adverse response or reaction;

ii) determining whether the subject has the marker associated with an adverse response or reaction; and optionally

iii) treating the subject with a therapeutic to ameliorate or prevent an adverse response or reaction.

Adverse reaction to metal debris (ARMD) or adverse local tissue reaction (ALTR), such as ALVAL, is a newly described, pathological response that can occur in response to implanted metal devices, which is frequently associated with significant pain, disability and irreversible tissue destruction. It is a poorly understood condition, but one which may affect hundreds of thousands of patients with metal implants throughout the world.

While the use of MoM hips has been greatly reduced in light of such complications, there is an increasing number of reports of ALVAL in MoP (metal on plastics) devices. It is possible that ARMD/ALTR may well be, and has been for decades, an under recognised source of unexplained pain and reduced patient satisfaction following joint replacement surgery. Improving the understanding of ARMD/ALTR is therefore important for the current management of at-risk patients, for the future development of prostheses and, possibly, the investigation and treatment of other immune mediated/inflammatory conditions. Furthermore, it may open a new avenue for personalised joint replacement guided by an individual's genetically determined reactivity to certain compounds. As an example, despite the negative publicity, MoM hip resurfacing has performed extremely well in younger active male patients.

The marker associated with ARMD/ALTR may comprise any one or more of the following:

HLA genotypes which are associated with common autoimmune inflammatory conditions.

The marker associated with ARMD/ALTR may comprise any one or more of the following such as Rheumatoid arthritis (eg HLA-DRB1*01, DRB1*04, DRB1*10(18, 19)), coeliac (DQA1*05:01/DQB1*02:01) and/or Crohn's disease (such as HLA-DRB1*07, HLA-DRB1*0103, HLA-DRB1*04 and HLA-DRB3*0301.(20)).

The marker associated with ARMD/ALTR may comprise any one or more of the following alleles: HLA-DRB1*01, DRB1*04, DRB1*10(18, 19), DQA1*05:01/DQB1*02:01, HLA-DRB1*07, HLA-DRB1*0103, HLA-DRB1*04 and HLA-DRB3*0301.(20).

The Applicant has determined that certain HLA genotypes (which Applicants term “proreactive”) which are associated with common autoimmune inflammatory conditions such as Rheumatoid arthritis (eg HLA-DRB1*01, DRB1*04, DRB1*10(18, 19)), coeliac (DQA1*05:01/DQB1*02:01) and Crohn's disease (such as HLA-DRB1*07, HLA-DRB1*0103, HLA-DRB1*04 and HLA-DRB3*0301.(20)), are associated with the development of ALVAL.

ALVAL is a lymphocyte driven response and is associated with CoCr alloys, rarely with non-cobalt containing components.

ALVAL can result in widespread, irreversible tissue damage.

When metal particles are liberated from prostheses they combine with carrier proteins to form metalloproteins. Metalloproteins are ingested by dendritic cells or macrophages in a process called “phagocytosis”. After phagocytosis, the particle is transported to a lysosome. There, it is acidified, and the particle fragments into its constituent peptide chains. At the lysosome, a structure called the major histocompatibility complex (MHC) can bind with one of these peptides. It does this at a part of its structure called the “peptide binding groove”.

After an MHC molecule has bound a fragment with sufficient stability, the MHC molecule and the newly bound peptide travel to the surface of the cell and “present” the peptide to the outside environment (for this reason, macrophages and dendritic cells are called “antigen presenting cells”). Here, any passing lymphocytes may bind this complex and then become activated, releasing cytokines and traveling to the source of antigen release. This lymphocyte “activation” is the key step in determining whether ALVAL develops.

The three dimensional structure of the peptide binding groove is critical in determining which peptides are “presented”. This structure is genetically encoded. Some individuals have genes which encode very different peptide binding grooves—thus some individuals will respond differently to different antigens.

The biological sample may comprise a solid and/or fluid sample. The fluid sample may be a blood sample, saliva or a blood extract sample. The biological sample may comprise skin cells from the buccal cavity, for example.

The method may be an in vitro method.

The method may be employed to be used prior to joint replacement to determine risk of patient developing ARMD/ALTR related failure and/or used for joints in situ, in combination with blood metal ion concentrations to determine future risk of ARMD/ALTR related failure.

In accordance with another aspect of the present invention, there is provided a method of screening for a compound for use in preventing or ameliorating the likelihood of adverse response to metal debris (ARMD) or adverse local tissue reaction (ALTR) in a subject, said method comprising the steps of identifying an compound that interferes with MHC mediated immune response.

In accordance with a further aspect of the present invention, there is provided a composition for use preventing or ameliorating the likelihood of adverse response to metal debris (ARMD) or adverse local tissue reaction (ALTR) in a subject.

The composition may comprise testosterone or a metabolic precursor thereof.

Preferably the sample is a whole blood sample.

The Applicant has reverse engineered thousands of explanted prostheses to determine the amount of wear that has occurred in the body. The total amount of material lost is termed “volumetric wear”.

The Applicants used existing software which computer models the resulting peptide binding groove shape if the DQA1/DQB1 genetic combination is inputted. The software predicted the strength of binding of various peptide fragments to each possible peptide binding groove combination. The Applicant's discovered that patients with genotypes suited to the N terminal peptide fragments of albumin were significantly more likely to develop ALVAL. Other genotypes were associated with pain in the presence of a macrophage only ARMD response.

Using regression statistical modeling, with patient age and sex as variables included, the Applicants have invented a means to estimate a patient's relative risk of developing ARMD (either macrophage dominated or lymphocyte dominated (ALVAL). This could be used post operatively for advising follow up strategy but also pre-operatively to guide implant selection.

HLA genes (specifically the class II genes DQA1/DQB1) can be collected using non-invasive methods, such as from a cheek swab.

It is highly likely that the patients mounting an ALVAL response develop antibodies to the “antigen”. Knowing that the antigen will have particular affinity to the genetically determined peptide binding grooves will permit the use of a blood test to determine whether ALVAL is active in at risk patients.

The methods of the present invention may comprise the use of enzyme-linked immunosorbent assay (ELISA).

The present invention will now be described, by way of example only, with reference to the accompanying examples and figures, in which:

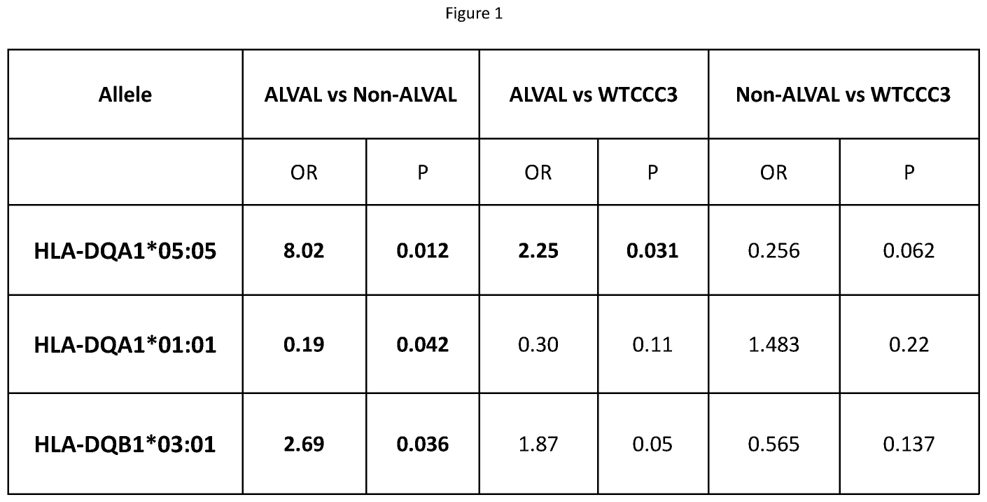

FIG. 1 is a table showing the result of experimentation to determine the affinity of certain alleles for ATCUN binder, MMBS binder and non-specific binding;

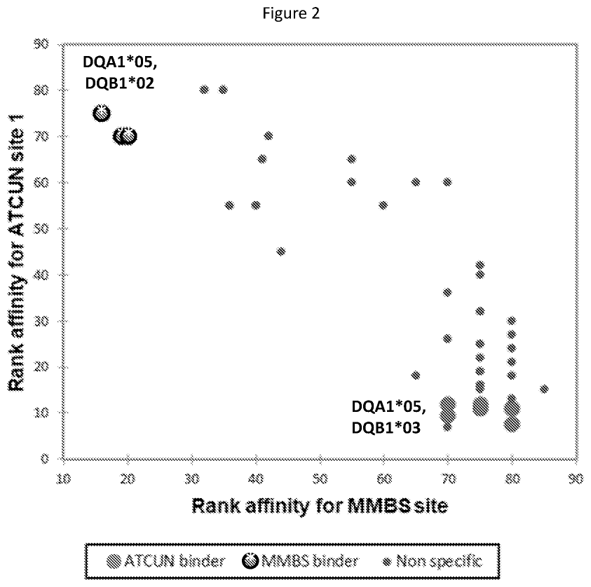

FIG. 2 shows the rank affinity for ATCUN and for MMBS;

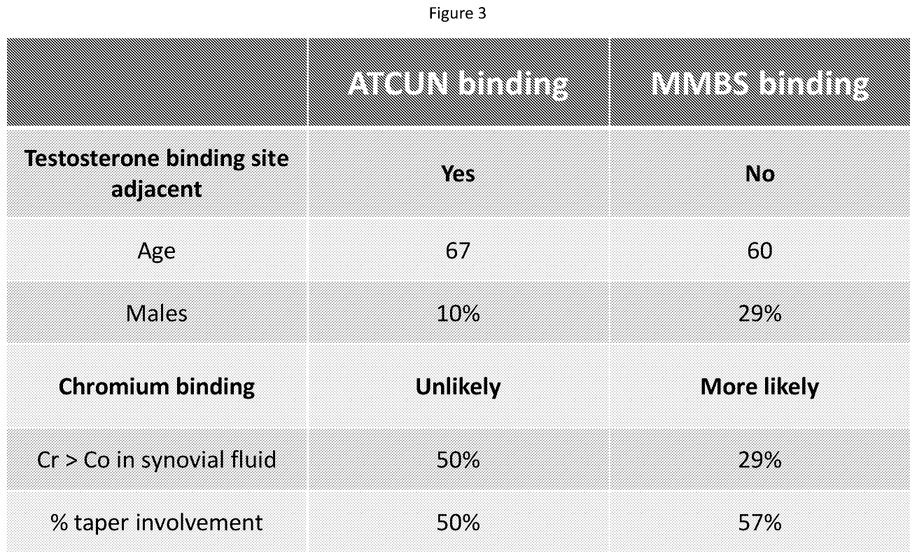

FIG. 3 is a table summarising the results of data collected from the data set; and

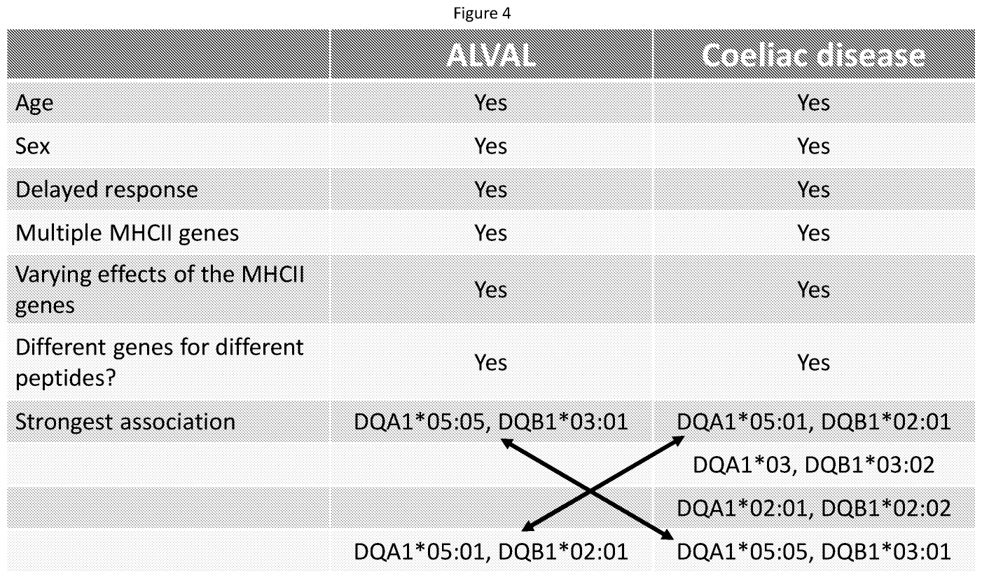

FIG. 4 is a table showing the correlation between likelihood of a ALVAL response and Coeliac disease markers.

### EXAMPLES

Excised tissues routinely undergo semiquantitative grading by a consultant histopathologist as previously described.(13, 17) From this pool of patients, three lists were created:

A group of patients with severe ALVAL with lowest wear rates (group A)

A group of patients with ALVAL exposed to high wear (group B)

A group of patients with no ALVAL findings (group C)

The HLA class I and class II genes of these patients were tested using next generation sequencing to a resolution of six digits.

The genotypes were compared between groups and with a background population of approximately 8500 patients from a previous study.

It became clear that certain DQA1/DQB1 combinations were more common in the group A patients compared to the group C and control group.

Identifying and isolating the group C patients with pain and low volumetric exposure wear showed an abnormal distribution of genetic haplotypes compared to the control population. These haplotypes were the same as those associated with the ALVAL response. This indicated that these patients were: 1. Captured prior to development of lymphocyte infiltration (ALVAL) or 2. that these genes are associated with hyperreactive macrophages, capable of causing adverse clinical sequelae without lymphocyte recruitment.

These combinations were entered into published software to determine which peptides the genetically encoded peptide binding grooves would bind with greater affinity.

The results indicate that there are certain, relatively common, genotypes which render a patient more susceptible to mounting a clinically adverse, lymphocytic response to metal debris. The alleles which showed the most obvious difference in distribution between patients who developed ALVAL, and those who did not, were found in the DQA1/DQB1 genetic loci. At the outset of this study, we focused on this area of the genome due to the clinical and pathological features that ALVAL shares with a number of inflammatory autoimmune/autoimmune like conditions, such as coeliac disease. The results appear to substantiate our hypothesis that these MHC molecules do indeed play a crucial role in the recruitment of lymphocytes in addition to the non-specific, largely macrophage dominated response to particulate debris.

The Immunogenetics of ALVAL

The Applicants focused on the divergence in the cellular response with reference to the development of ALVAL. We focused specifically on ALVAL for two major clinical reasons, the first being that the relationship between ALVAL and blood metal ion testing appears complex and ion concentrations do not appear to be of reliable diagnostic indicators in ruling the condition in or out. The second is that ALVAL appears to be an important, if not the most important, factor in the development of progressive and irreversible soft tissue damage.

The recruitment of lymphocytes to the area of macrophage driven inflammation lies in the handling and presentation of particulate debris to CD4+T cells by MHC II molecules on the membranes of APCs. Peptide-MHC binding is a prerequisite for T-cell immunogenicity and multiple studies have shown that there is a strong correlation between MHC peptide binding strength and peptide immunogenicity. Peptides that make stable peptide-MHC complexes accumulate on the cell surface and it has been shown that the total number of peptide-MHC complexes is important for T-cell activation.

When particulate matter is ingested by macrophages, peptide fragments which are produced in the lysosomes compete for the binding grooves of MHC molecules.

Applicants believe that that the antigen presented by APCs to initiate lymphocyte recruitment (the epitope) is a peptide derived from the breakdown of an albumin-Co metalloprotein. Further MHC II genes which differed in frequency between the ALVAL and non-ALVAL patient groups would show greater relative binding affinities for albumin derived peptides. Therefore, given the protection that younger age and male sex appears to confer, testosterone derived peptides might create competition with albumin for these binding sites.

Each patient has four potential DQA1/DQB1 combinations. It is therefore not ideal, when one is investigating the effect of a three-dimensional structure, to study only some of the building blocks in isolation i.e. by simply comparing alleleic frequencies of single genes between patient groups. By calculating theoretical binding affinities, produced a quantitative measure of a patient's genetic makeup which went beyond the classical approach of labeling a patient as being homozygous/heterozygous/lacking in an individual allele.

Applicants discovered that DQ molecules which are particularly suited to bind albumin fragments were significantly associated with the development of ALVAL. Conversely, DQ molecules which bind testosterone fragments with greater affinity were negatively associated with the development of ALVAL. Of particular significance, we believe, was that DQ isoforms in trans had relatively little influence on the statistical modeling compared to the cis combinations. Cis DQ isoforms are more easily formed, more numerous on cell membranes and are thought to be more important for T cell activation.

Albumin Binding and Antigen Presentation

Following antigen recognition and lymphocyte activation, chemokine release leads to the development of fluid exudates, with greater concentrations of albumin forming in the joint fluid. It is possible therefore that a vicious cycle is set in motion, with lymphocytes sensitised to an antigen which becomes present in ever greater quantities. An increase in the fraction of protein bound metal would also result in greater amounts of metal exiting the joint via the lymphatic system rather than via directly through the synovial membrane, as smaller solutes and ions are capable of doing.

Modulating Effects of Testosterone

Sex hormones play an important role in immune modulation. Furthermore, testosterone is present in the synovial fluid of healthy and arthritic joints in concentrations comparable to those of metal ions produced from low wearing MoM devices. A significant fraction of testosterone is albumin bound, meaning that particles digested by macrophages and dendritic cells are likely to contain varying amounts of Co, Cr, albumin and testosterone derived peptides. Levels of gonadal steroids in synovial fluid show an inverse relationship to age. Clearly, the modulation of the immune response by sex hormones is extremely complex. We suggest however that one of the reasons females and older patients may be more vulnerable to ALVAL might be due to their reduced synovial testosterone concentration and thus reduced competition for MHC binding spaces.

1. A method of screening a subject to determine the likelihood of adverse response to metal debris (ARMD) or adverse local tissue reaction (ALTR), said method comprising the steps of:
i) contacting in vitro a subject's biological sample with means to detect the presence and/or absence of a marker associated with an adverse response
ii) determining whether the subject has the marker associated with an adverse response
iii) treating the subject with a therapeutic to ameliorate an adverse response.

2. A method as claimed in claim 1 wherein the marker associated with adverse reaction to metal debris (ARMD) and/or adverse local tissue reaction (ALTR) comprises a HLA genotype associated with an autoimmune inflammatory condition.

3. A method as claimed in claim 2 wherein the inflammatory condition is selected from the group comprising rheumatoid arthritis, coeliac disease and/or Crohn's disease.

4. A method as claimed in claim 2 wherein the marker associated with adverse reaction to metal debris (ARMD) and/or adverse local tissue reaction (ALTR) comprises any one or more of the following: HLA-DRB1*01, DRB1*04, DRB1*10(18, 19), DQA1*05:01/DQB1*02:01, HLA-DRB1*07, HLA-DRB1*0103, HLA-DRB1*04 and HLA-DRB3*0301.(20).

5. A method as claimed in claim 1, 2, 3 or 4 wherein the biological sample comprises a solid and/or fluid sample.

6. A method as claimed in claim 5 wherein the fluid sample comprises one or more of the following:
a blood sample, saliva, skin cells or a blood extract sample.
A method as claimed in claim 3, wherein the biological sample comprises skin cells from the buccal cavity.

7. A method of screening a therapeutic agent for use in the treatment of adverse reaction to metal debris (ARMD) and/or adverse local tissue reaction (ALTR) response to an implant, said method comprising the steps of contacting the agent with albumin-cobalt metalloprotein to determine if binding occurs.

8. A method of screening as claimed in any one of the previous claims wherein the adverse reaction is ALVAL.

9. A method as claimed in any one of the previous claims for use in screening a subject prior to, during and/or after an implant procedure.

10. A method of treating a subject to prevent an adverse reaction to metal debris (ARMD) and/or adverse local tissue reaction (ALTR) to an implant comprising administering an agent to ameliorate or prevent an adverse reaction.

11. A compound for use in the treatment of a subject to prevent an adverse reaction to metal debris (ARMD) and/or adverse local tissue reaction (ALTR) to an implant.

12. A method of screening for a compound for use in preventing or ameliorating the likelihood of adverse response to metal debris (ARMD) or adverse local tissue reaction (ALTR) in a subject, said method comprising the steps of identifying an compound that interferes with MHC mediated immune response.
